(19) United States
(12) Patent Application Publication
(10) Pub. No.: US 2020/0298022 A1
(43) Pub. Date: Sep. 24, 2020

(54) Pencil Beam Therapy with Fast Deflection Magnet

(71) Applicant: Pyramid Technical Consultants Inc., Lexington, MA (US)

(72) Inventors: John Gordon, Henfield (GB); Andrew Dart, Swampscott, MA (US); Raymond Paul Boisseau, Waltham, MA (US)

(21) Appl. No.: 16/358,044

(22) Filed: Mar. 19, 2019

(51) Int. Cl. A61N 5/10

(52) U.S. Cl. CPC A61N 5/1043; A61N 5/1067; A61N 2005/1074; A61N 2005/1087; A61N 5/1075; A61N 5/103

(57) ABSTRACT

A pencil beam system includes a charged particle beam generator, a transport beamline apparatus, a scan nozzle, a fast deflector electromagnet, and a controller. After a therapeutic dose is delivered to a first target spot, the fast deflector electromagnet generates a first magnetic field that causes the net deflection of the charged particle beam to transition from the first target spot to an adjacent target spot. After the charged particle beam is directed to the adjacent target spot, the controller simultaneously adjusts the first magnetic field and the scan nozzle magnetic field to reduce and eliminate the contribution of the first magnetic field to the net deflection. The fast deflector electromagnet is deliberately designed with limited magnetic field and limited deflecting power to provide a higher slew rate, faster settling and less hysteresis contribution to beam position as compared to the scan nozzle electromagnets.

### TECHNICAL FIELD

This application relates to controlling pencil beam treatments and treatment systems, for example in the context of proton beam therapy and more particularly to methods for positioning.

### BACKGROUND

Charged particle therapy is used to treat certain conditions (e.g., cancer) in patients using focused, collimated or other spatially limited energetic particle beams. The principle generally relies on the controlled and localized deposition of sufficient dose of ionizing radiation in a treatment volume. The treatment volume may be an arbitrary three-dimensional volume (e.g., a cancer tumor) within the patient's body. In some instances, ionizing radiation is used to physically overcome the diseased tissue's survival thresholds and thereby destroy the diseased tissue.

In all such therapy procedures it is important to control the amount and location of the applied therapy beams and fields applied to a patient's body to avoid or minimize harm to healthy tissues and organs in the vicinity of the diseased volume. Treatment Planning Systems, sometimes employing medical imaging to guide the therapy procedure, are used to define the treatment volume and to prescribe the application of the therapy to the treatment volume. Time-dependent modeling, monitoring and other controls are employed to safely carry out proton therapy and similar treatments because the beams used in the treatments can accidentally injure the patients if applied incorrectly.

Pencil beam proton and other light ion therapy is used because of its ability to deliver dose to the patient with greatly improved spatial resolution and accuracy. It employs relatively narrow cross-sectional beams of protons, which can be on the order of a few millimeters in diameter. The advantages of the method require that the proton beam is positioned with a high degree of precision.

FIG. 1 illustrates a basic light ion therapy system such as a pencil beam proton therapy system (PBS) 10. Current proton therapy systems 10 include a proton beam source 100, which can generate a directed beam of ionizing radiation 101 at a desired energy level (typically 30 to 250 MeV). The beam 101 is transported from the source to the scanning system and dose measurement system 120 (“Nozzle”). The beam transport beamline 110 deflects the beam 101 as needed using one or more primary bending electromagnets 112, fine trim electromagnets 114 or other components, as well as scanner deflectors 122 in scan nozzle 120. The bending electromagnets 112 and/or fine trim electromagnets 114 can comprise quadrupole electromagnets, sextupole electromagnets, and/or electrostatic deflectors. One or more ion chambers (sometimes “IC”) 124 are disposed before the target of the beam 101. In some embodiments, two or more ICs 124 are disposed before the target of the beam 101. During therapy, the target is at a location or “spot” in a patient, but it is characterized for control purposes by its projection onto the nominal “isocenter” plane 105. The spots are defined in the treatment planning system (e.g., in irradiation maps) as having x and y positions (e.g., in mm), a beam energy (e.g., in MeV), a spot size in each of the x- and y-axes (e.g., defined as a sigma of a Gaussian-shaped beam along each axis) and a required dose.

During treatment, the beam is directed to multiple spot control points (defined on the isocenter plane 105) in the patient sequentially. The standard operating mode for such dose delivery is known as “Step & Shoot” where the system (a) set the scan magnets to point the beam to position X, Y corresponding to a spot control point, (b) turns on the beam, (c) monitors the dose until desired value is achieved, and (d) turns off the beam. This sequence is repeated for every spot control point in the irradiation map.

Depending on the system details, spot spacing, and magnet slew speed, the amount of time spent with the beam off can be excessive. For example, if the dose delivery requires an average of 5 ms per spot, but the process of positioning the beam requires an average of 10 ms, the dose is only being administered for 33% of the time. This results in long treatment times that are less comfortable for the patient and costly due to equipment use. Longer treatment also increases the risk of the shifting of internal organs, which can make treatment less effective and cause patient distress.

One solution to this problem is to leave the beam on and redefine the irradiation map so that it can be delivered in a more continuous manner, which has been incorporated in some systems. The most radical change is to allow the beam to move continuously with either or both of speed of movement and beam current intensity being modulated in real time. However this dose delivery method does not correspond well to the irradiation map, which assumes a set of specific control points. Ideally, it would be preferable to have this mode of delivery fully defined by the treatment planning system, but currently it is not an available feature. Additionally the performance requirements imposed on the scan system electronics and software for such a mode of delivery are significant.

Another system called the “raster scan” system, developed by GSI Darmstadt and used at HIT and at Siemens installations, can be considered an intermediate case. The raster scan system does not turn off the beam for the majority of spot moves, but does rest at control points, making the assumption that the dose that is delivered during the move can be allocated to the control locations at the start or end of the move. However the system relies on a very large scan-magnet-to-isocenter distance for its speed, which is not compatible with many system designs.

It would be desirable to overcome these and other deficiencies in current charged particle beam delivery system.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the disclosure, some of the advantageous features will now be summarized. Other objects, advantages and novel features of the disclosure will be set forth in the following detailed description of the disclosure when considered in conjunction with the drawings, which are intended to illustrate, not limit, the disclosure.

An aspect of the invention is directed to a system for increasing dose delivery efficiency during charged particle beam therapy, comprising: a charged particle beam generator to generate a charged particle beam; a transport beamline apparatus comprising beamline deflector magnets that generate magnetic fields to deflect the charged generated particle beam towards a scan nozzle parallel to a reference axis. The scan nozzle comprises a scan electromagnet that generates a scan magnetic field to deflect the charged particle beam along a trajectory to a first target position on an isocenter plane. The system further comprises a detector apparatus disposed between the at least one scan magnet and the isocenter plane, the detector apparatus configured to output a signal representing a measured position of the charged particle beam with respect to orthogonal first and second axes, the reference axis orthogonal to the first and second axes; and a fast deflector electromagnet assembly disposed between the transport beamline apparatus and the scan nozzle, the fast deflector electromagnet assembly configured to (a) receive a first control signal and (b) generate a first magnetic field based on the first control signal, the first magnetic field and the scan magnetic field providing a combined deflection of the charged particle beam to deflect the generated particle beam from a first trajectory corresponding to the first target spot on the isocenter plane to a second trajectory corresponding to a second target spot on the isocenter plane; wherein the control system comprises a processor, the control system configured to: receive as an input the first and second spot positions, determine a trajectory correction by comparing the first and second spot positions, and generate the first control signal based on the trajectory correction, and wherein an inductance of the fast deflector electromagnet assembly is lower than an inductance of the scan electromagnet.

In one or more embodiments, a slew rate of the fast deflector electromagnet assembly is higher than a slew rate of the scan electromagnet. In one or more embodiments, the fast deflector electromagnet assembly includes a fast single-axis deflector electromagnet assembly that only deflects the charged particle beam with respect to the first axis. In one or more embodiments, the controller is further configured to determine whether the second target spot is located in a preferred direction from the first target spot, the preferred direction parallel to the first axis.

In one or more embodiments, the control system is further configured to: determine when a predetermined dose is delivered to the first target spot, and send the first control signal to the fast electromagnet when the predetermined dose is delivered to the first target spot.

In one or more embodiments, the control system is further configured to: generate a second control signal based on the trajectory correction, determine when the measured position of the charged particle beam corresponds to the second target spot, and when the measured position of the charged particle beam corresponds to the second target spot: send the second control signal to the scan electromagnet to adjust the scan magnetic field to deflect a hypothetical charged particle beam from the first target spot to the second target spot, the hypothetical charged particle beam having the first trajectory, generate a third control signal that adjusts a power to the fast electromagnet such that the charged generated particle beam stays on the second target spot while the scan electromagnet adjusts the scan magnetic field according to the second control signal, and send the third control signal to the fast electromagnet. In one or more embodiments, the third control signal causes the fast deflector electromagnet to transition to an off state when the scan electromagnet has adjusted the scan magnetic field to deflect the hypothetical charged particle beam and the charged particle beam from the first target spot position to the second target spot.

In one or more embodiments, the fast deflector electromagnet assembly includes a fast dual-axis deflector electromagnet assembly that can deflect the charged particle beam with respect to the first axis, the second axis, or both the first and second axes. In one or more embodiments, the fast deflector electromagnet assembly includes a return yoke comprised of a non-conductive magnetic material having a bulk resistivity of at least about 0.1 Ωm. In one or more embodiments, the non-conductive magnetic material comprises a ferrite material. In one or more embodiments, the inductance of the fast deflector electromagnet assembly is about 75 μH to about 250 μH.

Another aspect of the invention is directed to a method for increasing dose delivery efficiency during charged particle beam therapy, the method comprising: (a) generating a scan magnetic field with a scan electromagnet to deflect a charged particle beam to a first target spot on an isocenter plane; (b) generating a second magnetic field with a fast deflector electromagnet, the scan magnetic field and the second magnetic field providing a combined deflection of the charged particle beam from a first trajectory corresponding to the first target spot to a second trajectory corresponding to a second target spot on the isocenter plane, the fast deflector electromagnet having a lower inductance than the scan electromagnet, the scan electromagnet disposed between the fast deflector electromagnet and the isocenter plane; (c) after step (b), simultaneously adjusting the scan magnetic field and the second magnetic field to reduce a contribution of the second magnetic field to the combined deflection of the charged particle beam; and (d) maintaining the second trajectory of the charged particle beam during step (c).

In one or more embodiments, step (c) further comprises decreasing a magnitude of the second magnetic field while increasing a magnitude of the scan magnetic field. In one or more embodiments, step (c) further comprises increasing a magnitude of the second magnetic field while decreasing a magnitude of the scan magnetic field.

In one or more embodiments, step (d) further comprises: detecting a detected position of the charged particle beam with at least one ion chamber detector disposed between the scan electromagnet and the isocenter plane; determining whether the detected position corresponds to the second target spot; and when the detected position does not correspond to the second target spot, adjusting the scan magnetic field, the second magnetic field, or both the scan magnetic field and the second magnetic field until the detected position corresponds to the second target spot.

In one or more embodiments, the method further comprises: (e) receiving an irradiation map that includes a location of the first and second target spots; and (f) determining whether the second target spot is located in a preferred direction from the first target spot, the preferred direction parallel to an axis of deflection of the fast deflector electromagnet. In one or more embodiments, the irradiation map includes the location of a third target spot on the isocenter plane, and the method further comprises: (g) determining whether the third target spot is located in the preferred direction from the second target spot; (h) when the third target spot is located in the preferred direction from the second target spot, generating a third magnetic field with the fast deflector electromagnet, the scan magnetic field and the third magnetic field providing a second combined deflection of the charged particle beam from the second trajectory corresponding to the second target spot to a third trajectory corresponding to the third target spot; and (i) when the third target spot is not located in the preferred direction from the second target spot, generating a second scan magnetic field with the scan electromagnet to deflect the charged particle beam from the second target spot to the third target spot.

In one or more embodiments, the method further comprises (j) after step (h), simultaneously adjusting the scan magnetic field and the third magnetic field to reduce a contribution of the third magnetic field to the second combined deflection; and (k) maintaining the third trajectory of the charged particle beam during step (j).

Yet another aspect of the invention is directed to a system for increasing dose delivery efficiency during charged particle beam therapy, the system comprising: a scan nozzle comprising: a scan electromagnet that generates a scan magnetic field to direct the charged particle beam along a trajectory to a target position on an isocenter plane; a detector apparatus disposed between the scan magnet and the isocenter plane, the detector apparatus outputting a signal representing a measured position of the charged particle beam with respect to orthogonal first and second axes, wherein the first and second axes are orthogonal to a reference axis; and a fast deflector electromagnet assembly configured to (a) receive a first control signal and (b) generate a first magnetic field based on the first control signal, the first magnetic field and the scan magnetic field providing a combined deflection of the charged particle beam that deflects the charged particle beam from a first trajectory corresponding to a first target spot on the isocenter plane to a second trajectory corresponding to a second target spot on the isocenter plane, wherein: the scan nozzle is disposed between the fast deflector electromagnet assembly and the isocenter plane, and an inductance of the fast deflector electromagnet assembly is lower than an inductance of the scan electromagnet.

In one or more embodiments, the scan nozzle receives a second control signal and the fast deflector electromagnet assembly receives a third control signal, the second and third control signals causing the scan nozzle and the fast deflector electromagnet assembly to simultaneously adjust the scan magnetic field and the first magnetic field, respectively, to reduce a contribution of the first magnetic to the combined deflection of the charged particle beam. In one or more embodiments, the inductance of the fast deflector electromagnet assembly is about 75 μH to about 250 μH and the fast deflector electromagnet assembly includes a return yoke comprised of a ferrite material.

### IN THE DRAWINGS

For a fuller understanding of the nature and advantages of the present disclosure, reference is made to the following detailed description of preferred embodiments and in connection with the accompanying drawings.

FIG. 1 illustrates a basic proton therapy system according to the prior art.

FIG. 2 illustrates a pencil beam system according to one or more embodiments.

FIG. 3 is a graphical representation of an irradiation map that includes target spot locations for a given beam energy, according to one or more embodiments.

FIG. 4A illustrates a simplified view of the pencil beam system of FIG. 2 to show the x component of the deflection of the actual beam and the hypothetical beam when the single-axis deflector electromagnet contributes to the beam deflection.

FIG. 4B illustrates a simplified view of pencil beam system of FIG. 2 to show the x component of the deflection of the actual beam after the deflection of the actual beam has fully transitioned back to the magnetic field generated by the X-scan deflector electromagnet, with no contribution from the fast single-axis deflector electromagnet.

FIG. 5 illustrates graphs of the total beam deflection over time with respect to a single axis and the contributions provided by the X-scan deflector electromagnet and the fast single-axis deflector electromagnet for an example deflection from a first target spot position having an “x” component of 20 mm to a second target spot having an “x” component at 30 mm.

FIG. 6 is a flow chart of a method for controlling a charged particle pencil beam to rapidly deliver a therapeutic dose at sequentially-delivered treatment spots, according to one or more embodiments.

FIG. 7 illustrates a pencil beam system according to an alternative embodiment.

FIG. 8 is a flow chart of a method for controlling a charged particle pencil beam to rapidly deliver a therapeutic dose at sequentially-delivered treatment spots, according to one or more embodiments.

FIG. 9 illustrates an example structure of a fast single-axis deflector, according to one or more embodiments.

FIG. 10 illustrates an example structure of a fast dual-axis deflector, according to one or more embodiments.

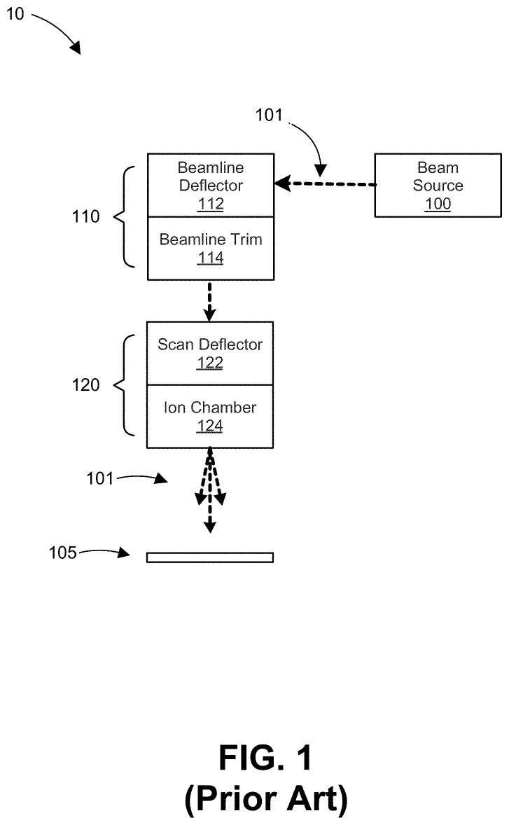

### DETAILED DESCRIPTION

A charged particle beam system includes a fast deflector electromagnet assembly disposed between the transport beamline apparatus and the scan nozzle. The fast deflector electromagnet assembly has a higher slew rate than the scan nozzle electromagnets due to several design features such as small gap field volume, low self-inductance due to a small number of coil turns, ferrite or powdered iron return yoke, and a deliberately-limited maximum deflection angle. Its deflection range is limited to approximately the typical distance between adjacent target spots on the isocenter plane.

In operation, the scan nozzle electromagnet directs or deflects the charged particle beam to a first target spot to deliver a therapeutic dose thereto. After the therapeutic dose is delivered, the fast deflector electromagnet assembly generates a magnetic field that, combined with the scan nozzle magnetic fields, deflects the charged particle beam from the first target spot to a neighboring second target spot. When the charged particle beam is redirected or deflected to the second target spot, after additional beam deflection by the fast deflector electromagnet assembly, dose delivery to the second target spot begins. Depending on the speed and slew rate of the fast deflector electromagnet assembly, the beam can be turned off between dose delivery at each spot (e.g., when speed/slew rate are relatively slow) or it can remain on (e.g., when speed/slew rate are relatively fast). After the charged particle beam is redirected to the second target spot, the control system causes the fast deflector electromagnet assembly and the scan electromagnets to simultaneously adjust their respective magnetic fields to reduce and eliminate the contribution of the magnetic field generated by the fast deflector electromagnet assembly to the combined deflection of the beam to the second target spot. After the deflection of the charged particle beam to the second target spot is fully transitioned to the scan magnets and the therapeutic dose is delivered to the second target spot, the fast deflector electromagnet assembly can be used again to deflect the charged particle beam from the second spot to a neighboring third target spot.

The higher slew rate and shorter settling time of the fast deflector electromagnet assembly allows the charged particle beam to be re-directed to the next target spot faster than would be possible by using the scan electromagnets by themselves while providing the control benefits of a Step & Shoot system. Thus, therapeutic dose delivery at the next target spot can occur sooner using the fast deflector electromagnet assembly than in prior system (e.g., using the scan electromagnet on its own), which increases therapeutic dose delivery efficiency.

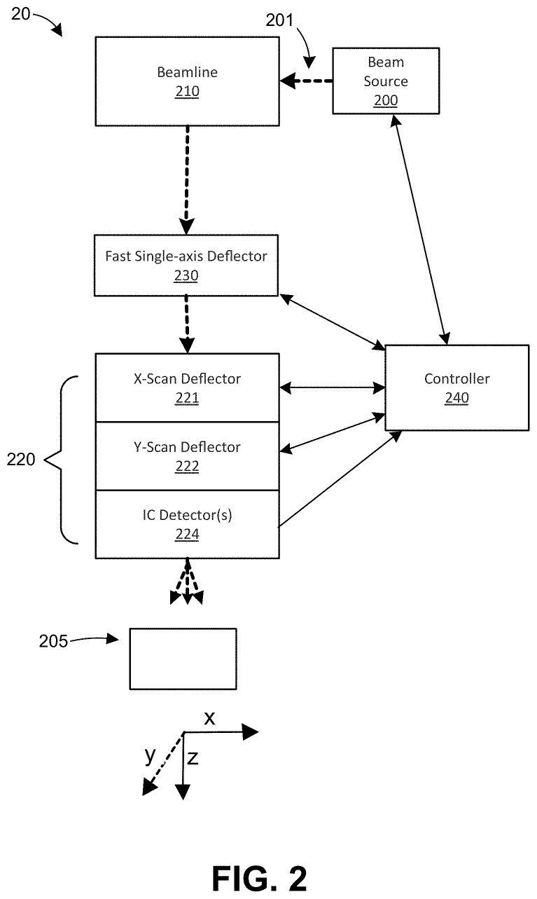

FIG. 2 illustrates a PBS 20 according to one or more embodiments. The system 20 includes beam source 200, beamline 210, scan nozzle 220, fast single-axis deflector electromagnet 230, and controller 240. Beam source 200, beamline 210, and scan nozzle 220 can be the same as or different than beam source 100, beamline 110, and scan nozzle 120, respectively, described above.

The scan nozzle 220 includes an X-scan deflector electromagnet 221, a Y-scan deflector electromagnet 222, and one or more ion chamber detectors 224. The X-scan deflector electromagnet 221 generates a magnetic field that deflects or bends beam 201 parallel to the “x” axis. Similarly, the Y-scan deflector electromagnet 222 generates a magnetic field that deflects beam 201 parallel to the “y” axis. The x and y axes are orthogonal to each other, and to reference axis “z,” which corresponds to the general direction of travel of beam 201 (e.g., as it passes from beamline 110 to the scan nozzle 220). For purposes of this disclosure, the “z” axis is static. However, it is noted that in beam optics the “z” axis refers to the trajectory of the beam 201, which can vary over its path.

The X-scan deflector electromagnet 221 and/or the Y-scan deflector electromagnet 222 can include a pair of dipole electromagnets that can deflect or bend the beam 201 parallel to the corresponding axis (e.g. parallel to the x and/or y axis). In some embodiments, the X- and Y-scan deflector electromagnets 221, 222 are the same as or different than scan deflector 122.

The IC detector(s) 224 measure the two-dimensional position of beam 201 after it passes through the X- and Y-scan deflector electromagnets 221, 222. The IC detector(s) 224 can include a first strip IC detector for measuring the position of charged particle pencil beam 201 with respect to a first axis (e.g., the x axis) and a second strip detector for measuring the position of beam 201 with respect to a second axis (e.g., the “y” axis). Such strip IC detectors include parallel rows of electrodes that can detect the position of the beam in a direction orthogonal to the orientation of the rows. For example, the first strip IC detector can have rows of electrodes that are oriented parallel to the y axis to detect the position of the beam with respect to the x axis. Similarly, the second strip IC detector can have rows of electrodes that are oriented parallel to the x axis to detect the position of the beam with respect to they axis. In other embodiments, the first and/or second strip IC detectors can have rows of electrodes that are not oriented parallel to the x and/or y axes. For example, the electrodes can be oriented, in the x-y plane, at a 30°, 45°, or 60° angle to the x and/or y axes.

The strip IC detectors are preferably thin to minimize scatter, energy degradation, and/or distortion of beam 201. An example of such a strip IC detector can be found in U.S. Pat. No. 9,293,310, titled “Method and Apparatus for Monitoring a Charged Particle Beam,” which is hereby incorporated by reference.

Alternatively, the IC detector(s) 224 can include a pixelated detector that includes a grid of electrodes (e.g., extending in both the x and y directions) to measure the position of beam 201 in a given plane (e.g., in the x-y plane, which is orthogonal to the z axis). An example of such a pixelated detector can be found in U.S. Pat. No. 9,731,149, titled “Method and Apparatus for Measuring, Verifying, and Displaying Progress of Dose Delivery in Scanned Beam Particle Therapy,” which is hereby incorporated by reference.

In some embodiments, the IC detector(s) 224 include one or more (e.g., a pair) of first strip IC detector for measuring the position and trajectory of the charged particle pencil beam 201 with respect to the x axis, and one or more (e.g., a pair) of second strip IC detector for measuring the position and trajectory of the charged particle pencil beam 201 with respect to the y axis. Alternatively, the IC detector(s) 224 can include a pair of pixelated detectors to determine the position and trajectory of the charged particle beam 201 with respect to both the x and y axes. In another embodiment, the IC detector(s) 224 can include a pixelated detector as a first IC detector and a first and second IC strip detectors for the x and y axes, respectively, as the second IC detector. The first IC detector can be disposed closer to the X- and Y-scan deflector electromagnets 221, 222 than the second IC detector. Alternatively, the second IC detector can be disposed closer to the X- and Y-scan deflector electromagnets 221, 222 than the first IC detector.

The IC detector(s) 224 outputs a signal to the controller 240 that represents the two-dimensional position of beam 201 at the location of the IC detector(s) 224. The signal is transmitted through high-speed readout electronics at a data rate that allows the controller 240 to react in a time comparable to the system response time (e.g., about 1 millisecond) to minimize any error in patient dosage. The controller 240 then determines the position (e.g., centroid) of the beam 201 based on the data (e.g., intensity distribution) provided in the output signal from the IC detector(s) 224. When the IC detector(s) 224 includes a pair of strip IC detectors, the controller 240 determines the position (e.g., with respect to the x or y axis) measured by each strip IC detector.

The controller 240 uses the two-dimensional position of the beam 201 at IC detector(s) 224 to determine the corresponding two-dimensional position of the beam 201 at isocenter plane 205. For example, the controller 240 can be calibrated to correlate the two-dimensional position of the beam 201 at IC detector(s) 224 with a projected two-dimensional position of the beam 201 at isocenter plane 205. In some embodiments, the controller 240 assumes a trajectory of the beam 201 to determine the projected two-dimensional position of the beam 201 at isocenter plane 250.

After the controller 240 determines the projected two-dimensional position of the beam 201 at isocenter plane 205, the controller compares the projected two-dimensional position of the beam 201 at isocenter plane 205 with a target two-dimensional position of the beam 201 at isocenter plane 205. The target two-dimensional position of the beam 201 can be provided in an irradiation map that includes a plurality of target two-dimensional positions or “spots.” For each spot, the irradiation map includes a target dose of energy to be deposited, a beam size, and a target energy of the beam 201. The target beam energy for proton therapy can be about 30 MeV to about 330 MeV (or more), preferably about 30 MeV to about 250 MeV, or any energy level between any of the foregoing beam energies.

If the projected two-dimensional position of the beam 201 at isocenter plane 205 is different (e.g., greater than an allowed tolerance range) than the target two-dimensional position of the beam 201 at isocenter plane 205, the controller 240 can generate control signals to adjust the magnetic field(s) generated by the X-scan deflector electromagnet 221 and/or the Y-scan deflector electromagnet 222 in a feedback loop until the projected two-dimensional position at isocenter plane 205 of the beam 201 is the same as, or within a predetermined tolerance range of, the target two-dimensional position at isocenter plane 205. In addition or in the alternative, the controller 240 can generate control signals to adjust the magnetic field(s) generated by the fast single-axis deflector electromagnet 230 to adjust the projected two-dimensional position at isocenter plane 205 of the beam 201 parallel to its preferred direction (e.g., parallel to the x- or y-axis).

After the target dose of energy is delivered to the target spot, the controller 240 generates a control signal that causes the scan nozzle 220 and/or the fast single-axis deflector electromagnet 230 to position the beam 201 at the next target spot and to deposit a target dose of energy, at a target beam energy, at that spot.

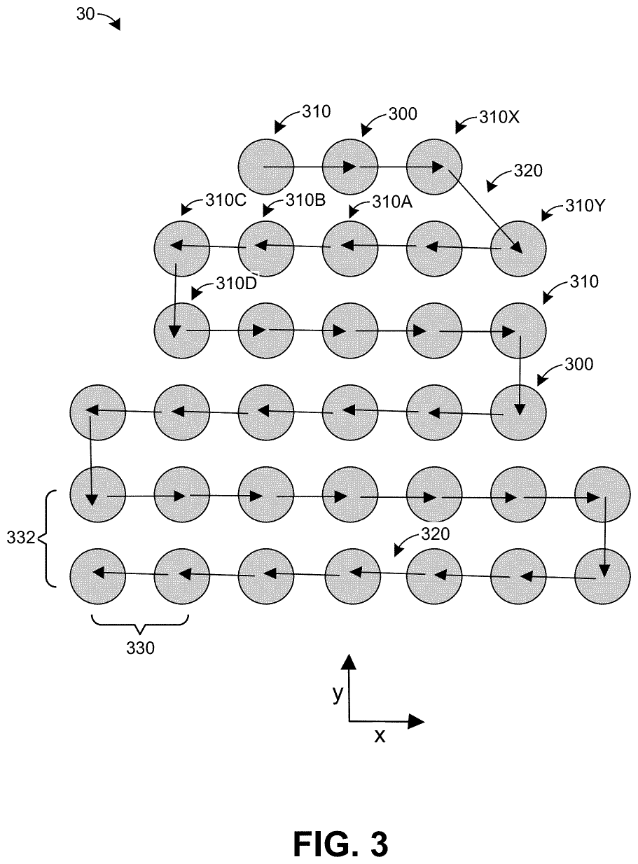

FIG. 3 is a graphical representation of an irradiation map 30 that includes target spot locations for a given beam energy, according to one or more embodiments. To maximize efficiency, the irradiation map 30 includes a sequential order of target spots for a given beam energy. The sequential order can be selected so that the physical distance between each target spot is minimized. For example, each target spot 300 in a grid can be located adjacent to or diagonally from a prior target spot 310 such that the beam follows a serpentine path 320. The physical distance between each adjacent target spot 300, which can be measured between the center of each target spot 300, can be about 2 mm to about 15 mm including about 4 mm, about 6 mm, about 8 mm, about 10 mm, about 12 mm, about 14 mm, or any distance or distance range between any two of the foregoing distances. The physical distance 330 between each adjacent target spot 300 for adjacent target spots 300 parallel to the x axis can be the same as or different than the physical distance 332 between adjacent target spots 300 parallel to they axis. When a larger jump between target spots 300 is needed, the fast single-axis deflector electromagnet 230 can be used to improve the settle time at the end of the jump, for example as disclosed in U.S. Pat. No. 10,183,178, titled “Method and Apparatus for Controlled Pencil Beam Therapy,” issued on Jan. 22, 2019, which is hereby incorporated by reference.

The serpentine path 320 can also be configured so that the majority of the beam travel is in a preferred direction, for example if one of the deflector electromagnets in scan nozzle 220 (e.g., the X-scan deflector electromagnet 221 or the Y-scan deflector electromagnet 222) is faster than the other, and/or to align with the preferred direction with the axis of deflection of the fast single-axis deflector electromagnet 230 (e.g., the x axis). In FIG. 3, the preferred direction is parallel to the x axis. In other embodiments, the preferred direction can be parallel to the y axis, for example when the fast single-axis deflector electromagnet 230 has an axis of deflection parallel to they axis. The serpentine path 320 has more target spot-to-target spot deflections or “hops” along the preferred direction (parallel to the x axis in FIG. 3) than it does along the non-preferred direction (parallel to the y axis in FIG. 3).

When the beam 201 needs to travel only in the preferred direction (e.g., the x axis) between spots, such as from a first target spot 310A to a second target spot 310B, the beam 201 is initially deflected by the fast single-axis deflector electromagnet 230. For example, the fast single-axis deflector electromagnet 230 initially provides an increase (or decrease) in deflection such that the combined deflection provided by the fast single-axis deflector electromagnet 230 and the X-scan deflector electromagnet 221 results in a beam trajectory that corresponds to the second target spot 310B

After the beam 201 is deflected to the second target spot 310B, the contribution to the combined deflection provided by the fast single-axis deflector electromagnet 230 is ramped down while contribution to the combined deflection provided by the X-scan deflector electromagnet 221 is ramped up. By ramping down the fast single-axis deflector electromagnet 230 and ramping up the X-scan deflector electromagnet 221 simultaneously, the beam 201 maintains its position second target spot 310B while the deflection of the beam 201 to the second target spot 310B is transitioned from the combined deflection from the fast single-axis deflector electromagnet 230 and the X-scan deflector electromagnet 221 back to deflection solely by the X-scan deflector electromagnet 221.

Since the fast single-axis deflector electromagnet 230 has a faster response rate (e.g., a higher slew rate) than the X-scan deflector electromagnet 221, the beam 201 is re-positioned from the first target spot 310A to the second target spot 310B at a faster rate by initially using the fast single-axis deflector electromagnet 230 (in combination with the X-scan deflector electromagnet 221) than by using the X-scan deflector electromagnet 221 on its own (e.g., in existing systems). After the deflection of the beam 201 to the second target spot 310B is transitioned back to the X-scan deflector electromagnet 221 and the target dose is delivered at the second target spot 310B, the above sequence can repeat. For example, the fast single-axis deflector electromagnet 230 can be used again to initially deflect the beam 201 from the second target spot 310B to a third target spot 310C.

In operation, the controller 240 generates a first control signal that causes the fast single-axis deflector electromagnet 230 to generate a magnetic field to deflect the beam 201 parallel to the x axis such that the beam 201 is deflected from the first target spot 310A to the second target spot 310B. The controller 240 can monitor the position of the beam using IC detector(s) 224 as feedback to adjust the first control signal, and thus the beam 201 position, accordingly. The first control signal can be based on pre-determined sequences of setting adjustments of the fast single-axis deflector electromagnet 230 based on prior calculations and measurements known to achieve the required fast change in position. Alternatively the first control signal can be based on the measured position or measured magnetic field in the scan magnet could be used as a control input. For example, if the relationship between beam spot position and magnetic field in the scan nozzle 220 electromagnets (e.g., X-scan deflector electromagnet 221, Y-scan deflector electromagnet 222) is well-known, then a gain factor can produce the required control signal for the fast single-axis deflector electromagnet 230. In another example, if the beam position can be measured fast enough, then a servo controller can stabilize the position as the scan nozzle 220 electromagnets changes by adjustments to the fast single-axis deflector electromagnet 230.

While the fast single-axis deflector electromagnet 230 provides a combined deflection with the X- and Y-scan deflector electromagnets 221, 222 to deflect the beam 201 from the first target spot 310A to the second target spot 310B, the X- and Y-scan deflector electromagnets 221, 222 continue to generate the same magnetic fields that deflected the beam 201 to the first target spot 310A before the fast single-axis deflector electromagnet 230 was turned on. In other words, the X- and Y-scan deflector electromagnets 221, 222 continue to generate magnetic fields that deflect a hypothetical beam 401 to the first target spot 310A. The hypothetical beam 401 has the same properties (e.g., energy, diameter, etc.) as the actual beam 201, but the hypothetical beam has the trajectory of the actual beam 201 before the fast single-axis deflector electromagnet was turned on.

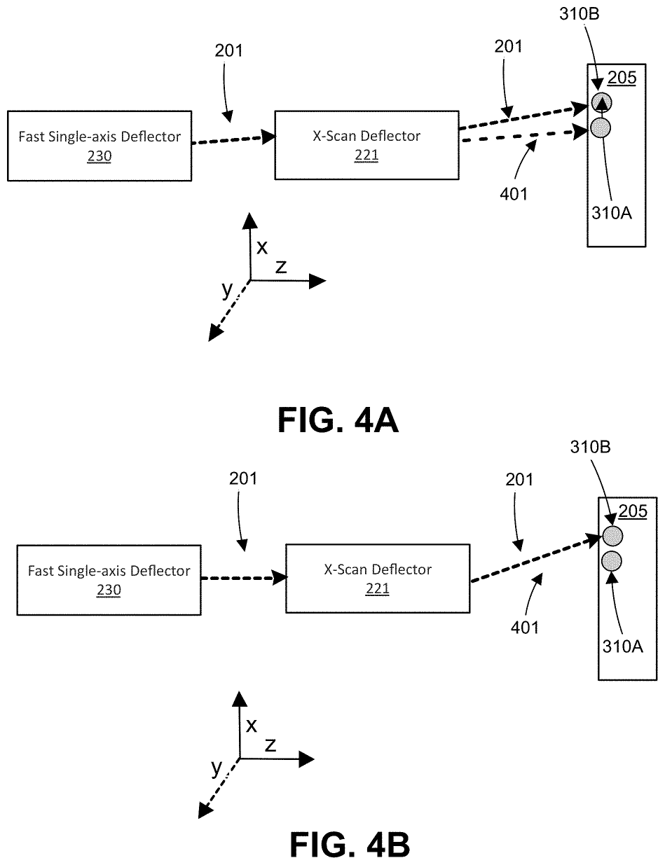

The magnetic field generated by the fast single-axis deflector electromagnet 230, in combination with the magnetic fields generated by the X- and Y-scan deflector electromagnets 221, 222, further deflects (or re-directs) the actual beam 201 to the second target spot 310B. FIG. 4A illustrates a simplified view of PBS 20 to show the x component of the deflection of the actual beam 201 and the hypothetical beam 401 when the single-axis deflector electromagnet 230 contributes to the beam deflection. The total deflection of the beam 201 parallel to the x axis is approximately equal to the sum of the deflection of the beam 201 by the X-scan deflector electromagnet 221 and of the deflection of the beam 201 by the fast single-axis deflector electromagnet 230. In FIG. 4A, the deflection of the beam 201 by the fast single-axis deflector electromagnet 230 is exaggerated for illustration purposes.

After the beam 201 is deflected to the second target spot 310B, the controller 240 generates a second control signal that causes the X-scan deflector electromagnet 221 to adjust (e.g., increase or decrease) the magnitude of its magnetic field such that the hypothetical beam 401 is deflected to the second target spot 310B. As the X-scan deflector electromagnet 221 adjusts (e.g., increases) its magnetic field, the controller 240 generates a third control signal that causes the fast single-axis deflector electromagnet 230 to adjust (e.g., decrease) its magnetic field so that the net deflection of the beam 201 remains constant and the beam 201 remains positioned on the second target spot 310B. As a result, the deflection of the beam to the second target spot 310B is fully transitioned from (a) a combination of the magnetic fields generated by the fast single-axis deflector electromagnet 230 and the X-scan deflector electromagnet 221 to (b) the magnetic field generated by the X-scan deflector electromagnet 221. FIG. 4B illustrates a simplified view of PBS 20 to show the x component of the deflection of the actual beam 201 after the deflection of the actual beam 201 has fully transitioned back to the magnetic field generated by the X-scan deflector electromagnet 221, with no contribution from the fast single-axis deflector electromagnet 230. FIG. 4B also illustrates that the hypothetical beam 401 is deflected to the second target spot 310B solely by the X-scan deflector electromagnet 221.

The advantage of adjusting the beam 201 trajectory parallel to the x axis (the preferred direction) from the first target spot 310A to the second target spot 310B using the fast single-axis deflector electromagnet 230 is that the fast single-axis deflector electromagnet 230 has a faster slew rate and a shorter settling time at the end of the slew due to negligible yoke saturation, minimal eddy currents (e.g., decaying eddy currents), and/or negligible mechanical change, than the X- and Y-scan deflector electromagnets 221, 222.

For example, the fast single-axis deflector electromagnet 230 can have a slew rate of about 50 to about 100 microseconds (or about 0.050 to about 0.10 milliseconds) to ramp to its target value. In contrast, the X-scan deflector electromagnet 221 (and/or the Y-scan deflector electromagnet 222) can have a slew rate of about 5 milliseconds to ramp to its target value and may also have an extended settling time to come within the desired tolerance. As such, the slew rate of the fast single-axis deflector electromagnet 230 can be about 5 to about 100 times higher than the slew rate of the X-scan deflector electromagnet 221 (and/or of the Y-scan deflector electromagnet 222). This means that the fast single-axis deflector electromagnet 230 can turn on its magnetic field to deflect the beam 201 from the first target spot 310A to the second target spot 310B about 5 to about 100 times faster than the time it takes the X-scan deflector electromagnet 221 (and/or the Y-scan deflector electromagnets 222) to turn on its magnetic field to cause the same deflection (from the first target spot 310A to the second target spot 310B). The increased slew rate of the fast single-axis deflector electromagnet 230 can also be used to correct for any imperfections in the X-scan deflector electromagnet 221 (or Y-scan deflector electromagnet 222 when the preferred direction of the fast single-axis deflector electromagnet 230 is parallel to the y axis).

The decreased settling time of the fast single-axis deflector electromagnet 230 is due, at least in part, to lower-magnitude and/or faster-decaying eddy current flows following slew of the fast single-axis deflector electromagnet 230 (e.g., due to the UR time constant of the eddy current circuit). The lower-magnitude and/or faster-decaying eddy current flows can have a negligible effect on the deflection of the beam 201 at isocenter 205. For example, the lower-magnitude and/or faster-decaying eddy current flows can deflect the beam 201, at isocenter 205, by about 0.1 mm or less or about 0.05 mm or less.

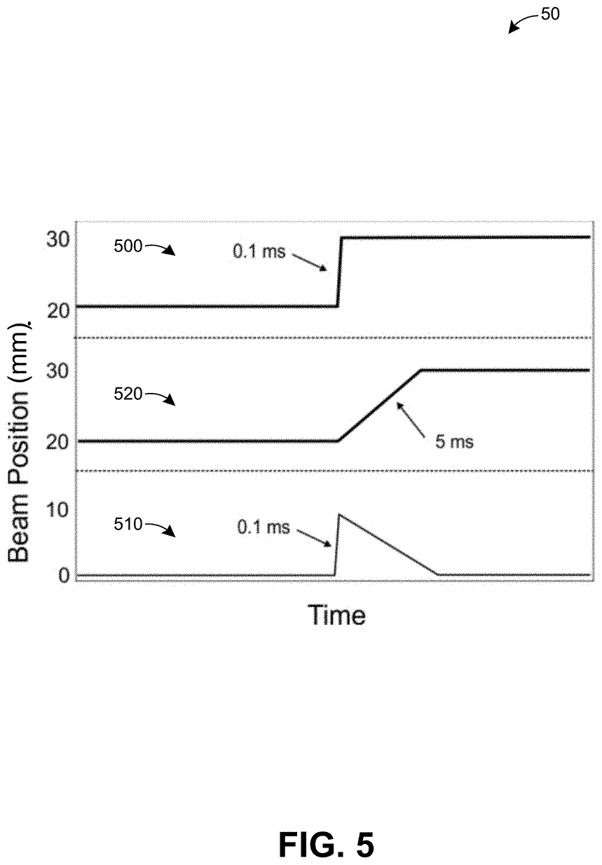

FIG. 5 illustrates graphs 50 of the total beam deflection over time with respect to a single axis and the contributions provided by the X-scan deflector electromagnet 221 and the fast single-axis deflector electromagnet 230 for an example deflection from a first target spot position having an “x” component of 20 mm to a second target spot having an “x” component at 30 mm. The graph 500 of the total or combined beam deflection over time illustrates that the beam position transitions from the first target spot (e.g., x=20 mm) to the second target spot (e.g., x=30 mm) in 0.1 milliseconds. This transition corresponds to the slew rate of the fast single-axis deflector electromagnet 230, as illustrated in graph 510. The X-scan deflector electromagnet 221 does not contribute to the beam deflection from the first target spot to the second target spot in the first 0.1 milliseconds after the fast single-axis deflector electromagnet 230 is turned on. For example, 0.1 milliseconds after the fast single-axis deflector electromagnet 230 is turned on, the fast single-axis deflector electromagnet 230 contributes about 10 mm of deflection parallel to the x axis and the X-scan deflector electromagnet 221 contributes about 20 mm of deflection parallel to the x axis, resulting in a total deflection of about 30 mm, which corresponds to the second target spot. A deflection of about 8 mm to about 15 mm parallel to the x axis can be the maximum deflection capability of the fast single-axis deflector electromagnet 230. In contrast, the X-scan deflector electromagnet can have a maximum deflection capability of about 150 mm to about 200 mm with respect to the x axis.

After the beam is deflected to the second target spot, the controller 240 causes the X-scan deflector electromagnet 221 to increase (or decrease) its magnetic field to increase the contribution of the X-scan deflector electromagnet 221 to the combined beam deflection from the first target spot to the second target spot. This increase (or decrease) occurs over 5 milliseconds, as illustrated in graph 520, which corresponds to the slew rate of the X-scan deflector electromagnet 221. In other embodiments, the slew rate of the X-scan deflector electromagnet 221 can be about 1 millisecond to about 20 milliseconds. At the same time, the controller 240 causes the fast single-axis deflector electromagnet 230 to decrease (or increase) its magnetic field to decrease the contribution of the fast single-axis deflector electromagnet 230 to the combined beam deflection from the first target spot to the second target spot. This increase (or decrease) occurs over 5 milliseconds, as illustrated in graph 510, so that the fast single-axis deflector electromagnet 230 and the X-scan deflector electromagnet 221 alter their magnetic fields, and their contribution to the combined beam deflection from the first target spot to the second target spot, simultaneously at the same rate. As a result, the total or combined beam deflection over time after the initial 0.1 millisecond transition, from the fast single-axis deflector electromagnet 230, remains constant at the second target spot (about 30 mm), as illustrated in graph 500.

The synchronization between the fast single-axis deflector electromagnet 230 and the X-scan deflector electromagnet 221 can be performed, to a good approximation, through synchronization of the respective output control waveforms. Improved performance can be achieved by monitoring the magnetic field generated by the X-scan deflector electromagnet 221 and driving the faster-responding fast single-axis deflector electromagnet 230 to precisely track and negate the ramp of the magnetic field generated by the X-scan deflector electromagnet 221.

Though the above discussion assumes that the quiescent point of the fast single-axis deflector electromagnet 230 is zero or “off,” it is noted that in other embodiments the quiescent point can be placed at the full-power value of the fast single-axis deflector electromagnet 230 (which would be compensated for by the scan electromagnets). In this embodiment, the fast single-axis deflector electromagnet 230 can be powered up to the full-power value at the opposite polarity to the quiescent point, which can increase the deflection power of the fast single-axis deflector electromagnet 230 by a factor of 2 compared to when the quiescent point is at zero or “off.”

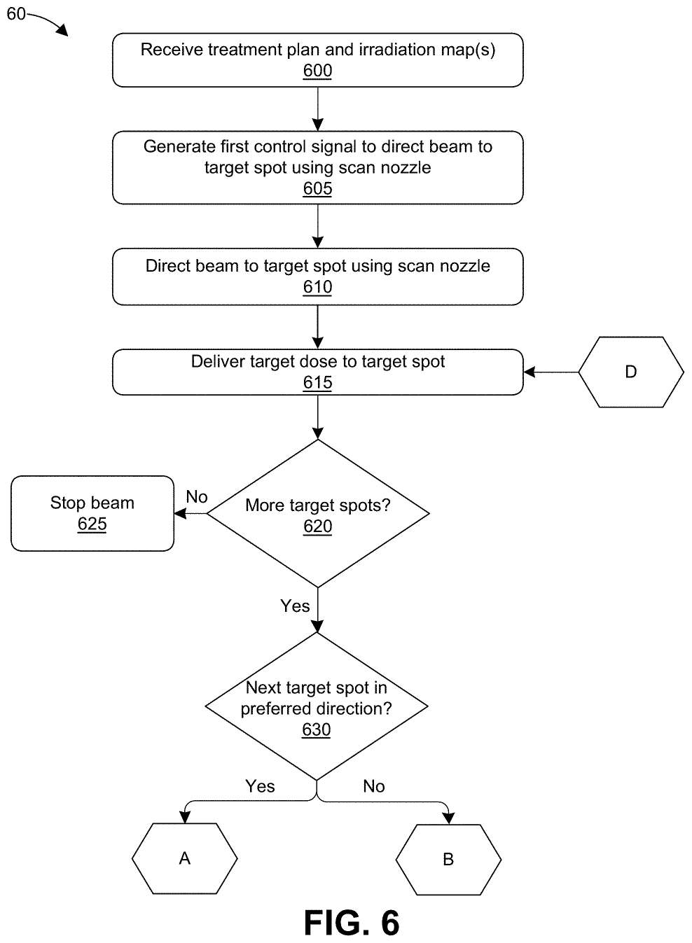

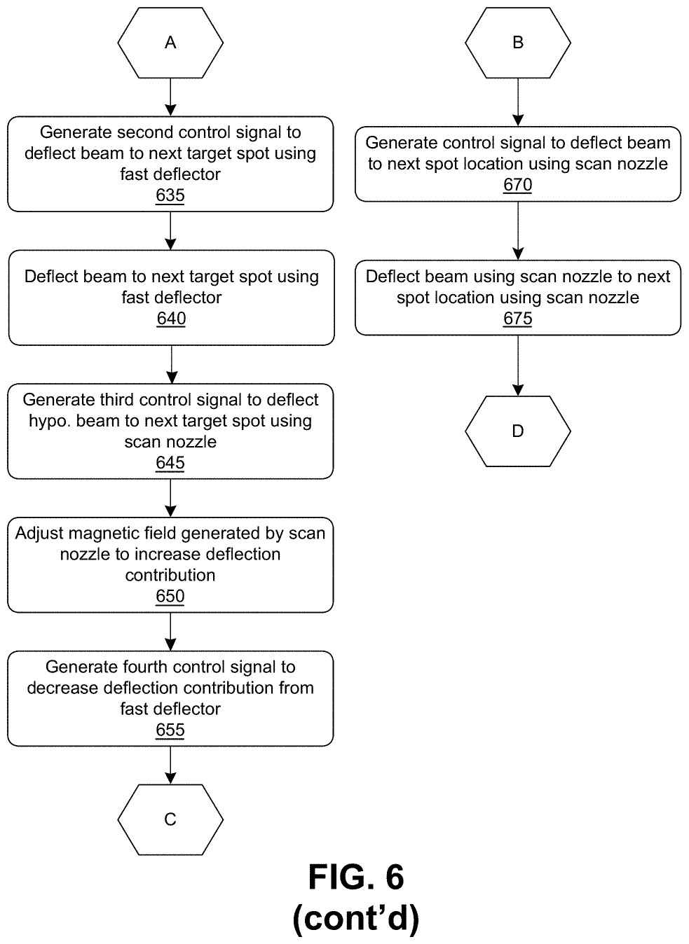

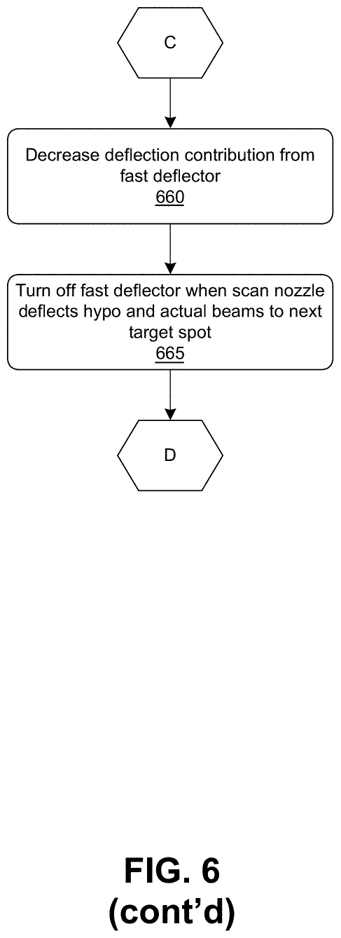

FIG. 6 is a flow chart of a method 60 for controlling a charged particle pencil beam to rapidly deliver a therapeutic dose at sequentially-delivered treatment spots, according to one or more embodiments. The method 60 begins at step 600 where a controller (e.g., controller 240) receives a treatment plan which can be stored in and/or created by a computer (e.g., a personal computer, a server, or other microprocessor-based computer) in network communication with the controller. The treatment plan includes the physical treatment locations in the patient, which can correspond to a three-dimensional anatomical feature in the patient such as an organ, a malignant tumor, or other anatomical feature. The treatment plan also includes the target total distribution of therapeutic dose to the physical treatment locations, and how the target total dose distribution will be delivered over multiple dose fractions, which can include from multiple beam angles. The controller also receives irradiation maps (sometimes referred to as scan maps) which are generated based on the treatment plan. The irradiation maps include, for each beam energy, target spot positions and size and the sequence that the target spots are to be delivered in a given irradiation. The target spots are defined with respect to the isocenter plane for purposes of the computer and controller. At a given beam energy, the target spots can be arranged in a grid-like manner on the plane, for example as illustrated in FIG. 3. The sequence of spots can provide a preferential direction for moving the beam (e.g., primarily parallel to the x or y axis) which can result in moving the beam along a serpentine-like path (e.g., serpentine path 320 in FIG. 3) for at least a portion of a given irradiation.

In step 605, the controller generates a first control signal that causes the scan nozzle to deflect the beam to a target spot (e.g. first target spot 310A). The scan nozzle can deflect the beam using one or more deflector electromagnets, such as X-scan deflector electromagnet 221 and Y-scan deflector electromagnet 222. In other embodiments, the scan nozzle can include a multipole electromagnet that can deflect the beam with respect to both the x and y axes.

In step 610, the scan nozzle generates a magnetic field in response to the received first control signal. The magnetic field directs the beam to the target spot. In some embodiments, the controller can receive feedback data from an IC detector disposed between the scan nozzle and the isocenter plane. The feedback data can correspond to the detected beam location (e.g., centroid position) of the beam at the location of the IC detector. The controller can compare the detected beam location with a model beam location at the location of the IC detector, and it can adjust the first control signal if there's at least a predetermined position error between the detected beam location with a model beam location. Alternatively, the controller can determine a projected beam location at isocenter plane based on the detected beam location at the location of the IC detector, such as by assuming the trajectory of the beam. The controller can then compare the projected beam location with the location of the target spot, and it can adjust the first control signal if there's at least a predetermined position error between the projected beam location and the location of the target spot.

In addition or in the alternative, the first control signal can be based on the measured magnetic field in the scan magnet could be used as a control input. For example, if the relationship between beam spot position and magnetic field in the scan nozzle electromagnets (e.g., X-scan deflector electromagnet 221, Y-scan deflector electromagnet 222) is well-known (e.g., based on prior offline measurements, calculations, and stored data), then this relationship can be used to generate the first control.

In step 615, the target dose of particular beam energy is delivered to the target spot. The target dose is included in the irradiation map, derived from the treatment plan, as discussed above. The controller can determine whether the target dose is delivered based on the amount of charge measured by the ionization chambers.

In step 620, the controller determines whether there are more target spots (e.g., according to the irradiation map) that need to receive a dose at the current energy level of the beam. If not, the controller stops the beam in step 625. The beam can then be adjusted to another energy level or treatment can be stopped. If there are more target spots in step 620, flow chart 60 proceeds to step 630.

In step 630, the controller determines whether the next target spot (e.g., a second target spot) is located in a preferred direction from the last target spot (e.g., the first target spot). The sequential order of target spots can be included in the irradiation map or it can be determined by the controller.

When the next target spot is located in a preferred direction from the last target spot (step 630=yes), such as the location of second target spot 310B with respect to first target spot 310A (discussed above), flow chart 60 proceeds to step 635 (via placeholder A). In step 635, the controller generates a second control signal that causes a fast single-axis deflector electromagnet (e.g., fast single-axis deflector electromagnet 230) to generate a magnetic field that causes a combined deflection of the beam (i.e., the combination of the magnetic fields generated by the fast single-axis deflector electromagnet and by the scan nozzle electromagnets) to the next target spot (e.g., second target spot 310B).

In step 640, the fast single-axis deflector electromagnet generates a magnetic field in response to the received second control signal. The fast single-axis deflector electromagnet has a high slew rate (e.g., about 0.050 to about 0.10 milliseconds), compared to the scan nozzle (e.g., compared to the X-scan deflector electromagnet 221 and the Y-scan deflector electromagnet 222), so that it can achieve the desired magnetic field rapidly (e.g., in about 0.050 to about 0.10 milliseconds). The magnetic field generated by the fast single-axis deflector electromagnet, in combination with the magnetic field(s) generated by the scan nozzle, directs the beam to the next target spot. The controller can receive feedback data from the IC detector disposed between the scan nozzle and the isocenter plane to adjust the second control signal, for example following an equivalent feedback loop as described above in step 610.

In step 645, the controller generates a third control signal to deflect a hypothetical beam from the last target spot (e.g., the first target spot) to the next target spot (e.g., the second target spot) using the scan nozzle. As discussed above, the hypothetical beam corresponds to the trajectory of the beam before the fast single-axis deflector electromagnet was turned on in step 640. In other words, the hypothetical beam has the same trajectory as the actual beam during steps 610 and 615.

In step 650, the scan nozzle adjusts (increases or decreases) its magnetic field in response to the received third control signal. The adjustment to the magnetic field generated by the scan nozzle deflects the hypothetical beam from the last target spot (e.g., the first target spot) to the next target spot (e.g., the second target spot). In addition, the adjustment to the magnetic field generated by the scan nozzle further deflects the actual beam. However, the deflection of the actual beam due to the adjustment to the magnetic field generated by the scan nozzle is offset by simultaneously adjusting the magnetic field generated by the fast single-axis deflector electromagnet such that the combined deflection of the beam remains constant. As a result, the adjustment to the magnetic field generated by the scan nozzle increases the deflection contribution from the scan nozzle (e.g., as illustrated in FIG. 5) to the combined deflection of the actual beam.

In step 655, the controller generates a fourth control signal that causes the fast single-axis deflector electromagnet to decrease the magnitude of its magnetic field to offset the adjustment to the scan nozzle's magnetic field in step 650. The adjustment to the magnetic field generated by the fast single-axis deflector electromagnet decreases the deflection contribution from the fast single-axis deflector electromagnet (e.g., as illustrated in FIG. 5) to the combined deflection of the actual beam, which occurs in step 660 (via placeholder C). Steps 650 and 660 occur simultaneously (or substantially simultaneously), for example as illustrated in FIG. 5, so that the adjustments to the magnetic fields generated by the scan nozzle and by the fast single-axis deflector electromagnet substantially offset one another (e.g., the spot position is maintained within a certain tolerance while delivering the dose, such as within a tolerance of ±about 0.5 mm or lower). There is no change in the net deflection of the beam and it continues to be directed to the next target spot (e.g., the second target spot).

In step 665, the fast single-axis deflector electromagnet is turned off when the scan nozzle has adjusted its magnetic field such that both the hypothetical beam and the actual beam are deflected to the next target spot (e.g., the second target spot) without any deflection contribution from the fast single-axis deflector electromagnet. When this occurs, the deflection of the beam to the next target spot has fully transitioned from a combination of the fast single-axis deflector electromagnet and the scan nozzle back to the scan nozzle on its own.

As can be seen, the method proceeds by first delivering a target dose of therapeutic energy at a first spot position. Once the target dose is delivered to the first spot position, the fast single-axis deflector electromagnet generates a magnetic field to deflect the beam to a second spot position that is adjacent to the first spot position and along the preferred direction of the fast single-axis deflector electromagnet. When the beam is deflected to the second spot position by the fast single-axis deflector electromagnet, the dose begins to be delivered to the second spot position. Next, the magnetic field generated by the scan nozzle is adjusted to initiate the same position change (from the first spot position to the second spot position) as that provided by the fast single-axis deflector electromagnet. While the scan nozzle's magnetic field is adjusted, the fast single-axis deflector electromagnet's magnetic field is simultaneously adjusted (e.g., relaxed) to compensate for the beam motion at the isocenter plane that would occur by the adjustment of the scan nozzle's magnetic field. In other words, the beam stays at the second spot position while the scan nozzle's magnetic field and the fast single-axis deflector electromagnet's magnetic field are adjusted. When the scan nozzle's magnetic field is fully adjusted, the fast single-axis deflector electromagnet's magnetic field is turned off (or returned to a “home” position). The adjustment of the fast single-axis deflector electromagnet's magnetic field during the adjustment of the scan nozzle's magnetic field can be made using a pre-programmed profile (e.g., based on prior calculations and measurements known to achieve the required change in position) or by using feedback information such as the beam's position (e.g., as measured by one or more ion chambers) and/or the magnetic field generated by the scan nozzle.

After step 665, flow chart 60 returns to step 615 (via placeholder D) where the target dose is delivered.

Returning to step 630, when the next target spot is located in a non-preferred direction from the last target spot (step 630=no), such as the location of the fourth target spot 310D with respect to third target spot 310C in FIG. 3, flow chart 60 proceeds to step 670 (via placeholder B). In step 670, the controller generates a fifth control signal that causes the scan nozzle (e.g., Y-scan deflector electromagnet 222) to deflect the beam to the next target spot.

In step 675, the scan nozzle alters (e.g., increases or decreases) its magnetic field in response to the received fifth control signal. The change to the magnetic field generated by the scan nozzle deflects the beam from the prior spot location (e.g., the third target spot 310C) to the next spot location (e.g., the fourth target spot 310D). After the beam is deflected to the next spot location, flow chart returns to step 615 (via placeholder D) where the target dose is delivered.

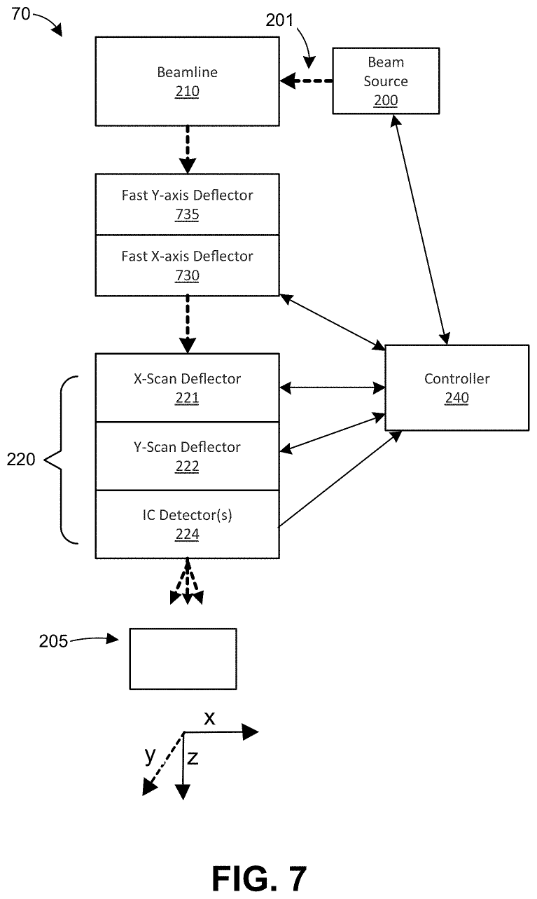

FIG. 7 illustrates a PBS 70 according to an alternative embodiment. PBS 70 is the same as PBS 20 except as described below. Instead of including a single fast single-axis deflector electromagnet (e.g., fast single-axis deflector electromagnet 230 in PBS 20), PBS 70 includes two fast single-axis deflector electromagnets: fast X-axis deflector electromagnet 730 and fast Y-axis deflector electromagnet 735. Fast X-axis deflector electromagnet 730 is the same as fast single-axis deflector electromagnet 230 when the fast single-axis deflector electromagnet 230 is configured to deflect the beam parallel to the x axis. Fast Y-axis deflector electromagnet 735 is the same as fast single-axis deflector electromagnet 230 when the fast single-axis deflector electromagnet 230 is configured to deflect the beam parallel to the y axis. In an alternative embodiment, fast X-axis deflector electromagnet 730 and fast Y-axis deflector electromagnet 735 are combined in a fast dual-axis deflector electromagnet.

As a result, PBS 70 can provide the same advantages as PBS 20 but with respect to two preferred directions: the x axis (or parallel to the x axis) and the y axis (or parallel to the y axis). This allows PBS 70 to provide the same advantages as PBS 20 when the spots in the irradiation maps are not arranged in straight rows along a preferred axis. To execute irradiation map 30, PBS 70 can transition between the first target spot 310A and the second target spot 310B (and between the second target spot 310B and the third target spot 310C) using fast X-axis deflector electromagnet 730 and it can transition between the third target spot 310C and the fourth target spot 310D using fast Y-axis deflector electromagnet 735. In some embodiments, PBS 70 can also transition diagonally between target spots 310X and 310Y in irradiation map 30 using both fast X-axis deflector electromagnet 730 and fast Y-axis deflector electromagnet 735.

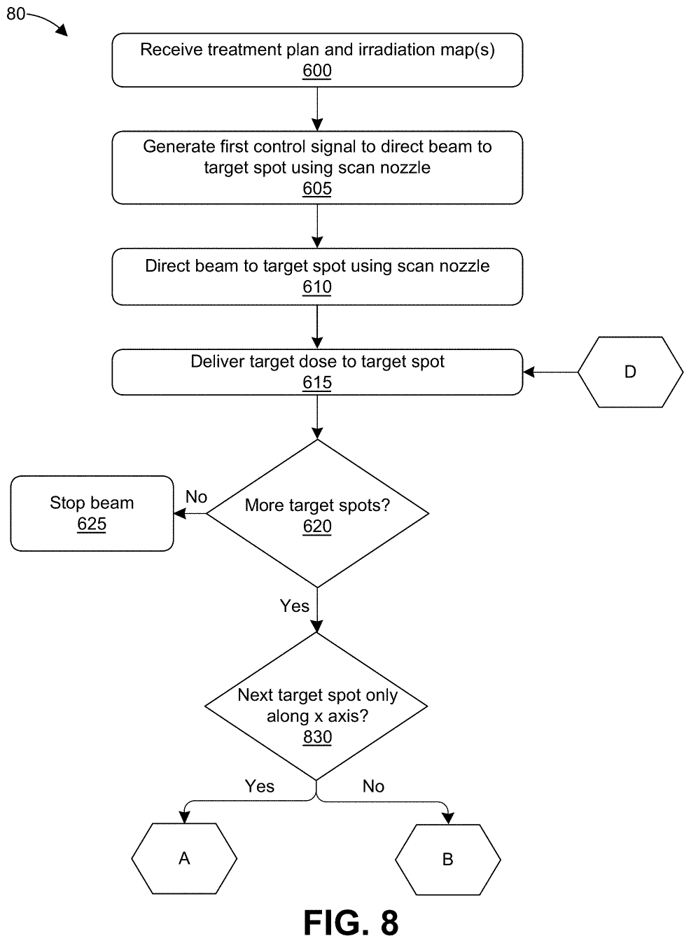

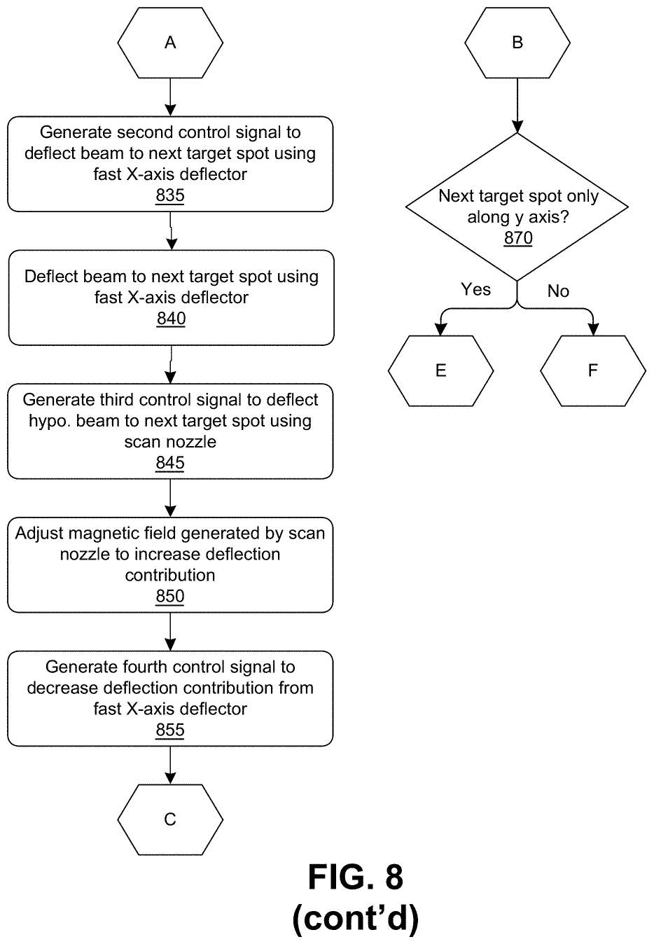

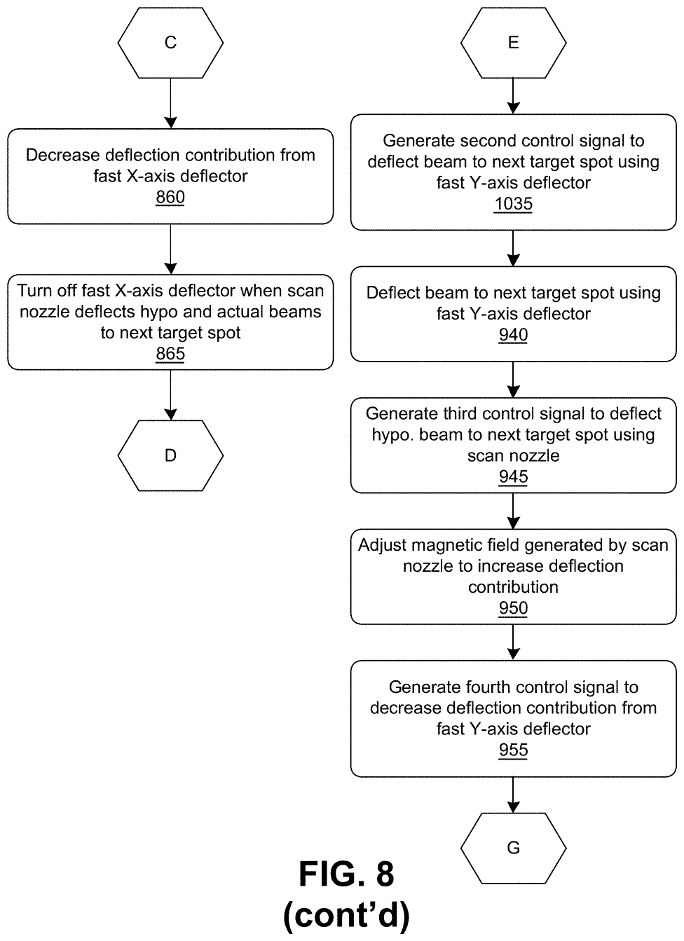

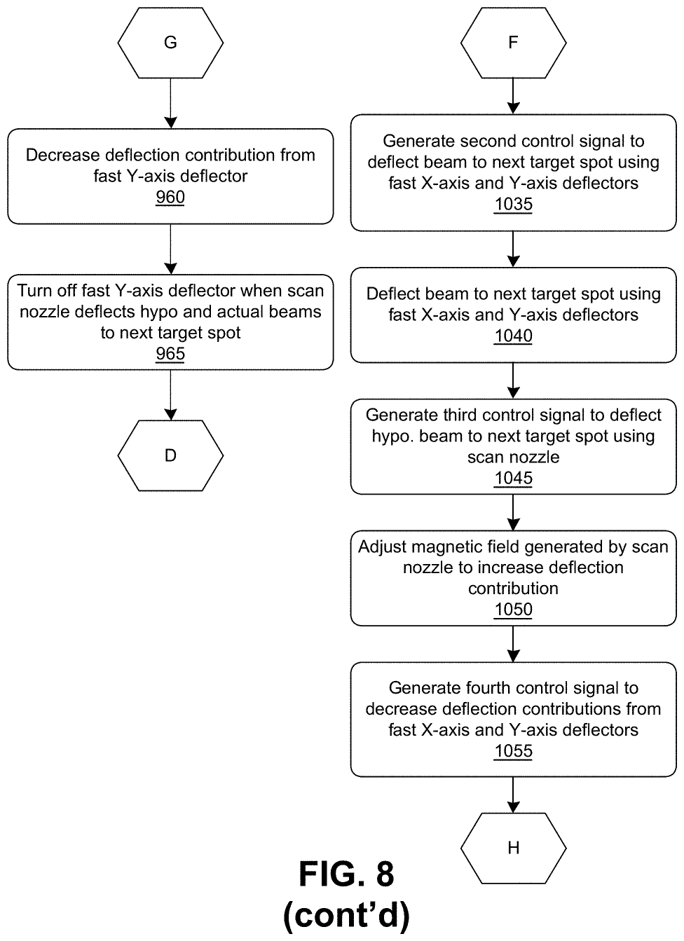

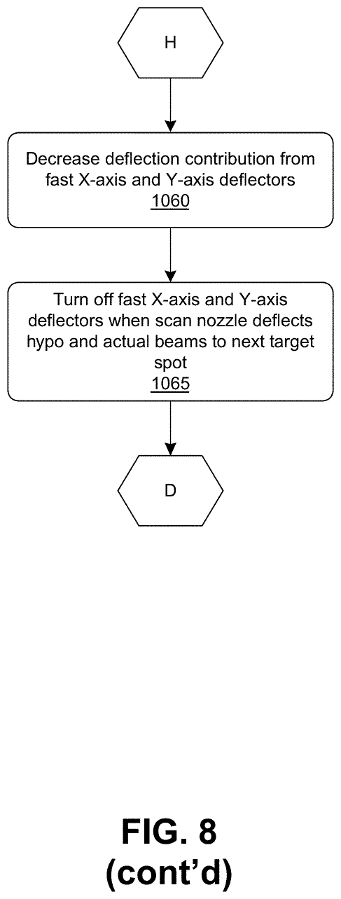

FIG. 8 is a flow chart 80 of a method for controlling a charged particle pencil beam to rapidly deliver a therapeutic dose at sequentially-delivered treatment spots, according to one or more embodiments. Flow chart 80 can be performed using PBS 70.

Steps 600, 605, 610, 615, 620, and 625 in flow chart 80 are the same as in flow chart 60.

In step 830, the controller determines whether the next target spot is located only parallel to the x axis from the last target spot (e.g., as in the first and second target spots 310A, 310B in FIG. 3).

When the next target spot is located only parallel to the x axis from the last target spot (step 830=yes), such as the location of the second target spot 310B with respect to the first target spot 310A, flow chart 80 proceeds to step 835 (via placeholder A). Steps 835, 840, 845, 850, 855, 860, and 865 are the same as steps 635, 640, 645, 650, 655, 660, and 665, respectively, when the preferred direction in flow chart 60 is parallel to the x axis.

When the next target spot is not located only parallel to the x axis from the last target spot (step 830=no), flow chart 80 proceeds to step 870 where the controller determines whether the next target spot is located only parallel to the y axis from the last target spot (e.g., as in the third and fourth target spots 310C, 310D in FIG. 3). When the next target spot is located only parallel to the y axis from the last target spot (step 870=yes), such as the location of the fourth target spot 310D with respect to the third target spot 310C, flow chart 80 proceeds to step 935 (via placeholder E). Steps 935, 940, 945, 950, 955, 960, and 965 are the same as steps 635, 640, 645, 650, 655, 660, and 665, respectively, when the preferred direction in flow chart 60 is parallel to the y axis.

When the next target spot is not located only parallel to the y axis from the last target spot (step 870=no) (e.g., it is located diagonally), such as the location of the target spot 310Y with respect to target spot 310X, flow chart 80 proceeds to step 1035 (via placeholder F). Steps 1035, 1040, 1045, 1050, 1055, 1060, and 1065 are the same as steps 635, 640, 645, 650, 655, 660, and 665, respectively, except that two fast single-axis deflectors are used to transition to the beam to the next spot in steps 1035, 1040, 1045, 1050, 1055, 1060, and 1065 instead of one fast single-axis deflector being used to transition to the beam to the next spot in steps 635, 640, 645, 650, 655, 660, and 665. In steps 1035, 1040, 1045, 1050, 1055, 1060, and 1065, the fast X-axis deflector transitions the x component of the diagonal deflection from the prior target spot to the next target spot and the fast Y-axis deflector transitions the y component of the diagonal deflection from the prior target spot to the next target spot. The transition back to deflection only by the scan nozzle in steps 1045, 1050, 1055, 1060, and 1065 occurs by adjusting the magnetic fields generated by the X-scan deflector electromagnet 221 and the Y-scan deflector electromagnet 222, or by adjusting the magnetic field generated by a multipole electromagnet that can deflect the beam in both the x and y directions.

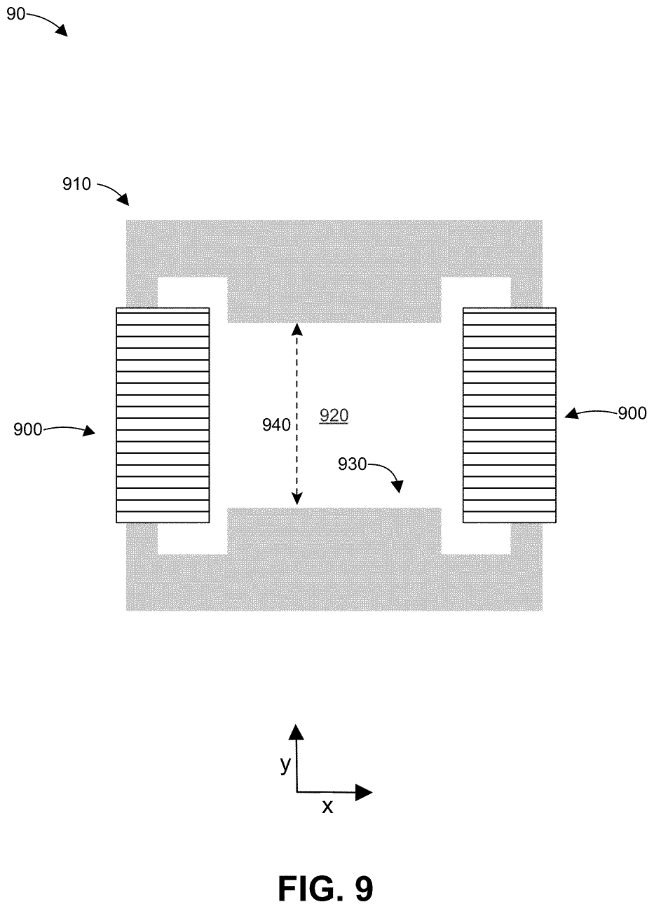

FIG. 9 illustrates an example structure of a fast single-axis deflector 90, according to one or more embodiments. The fast single-axis deflector 90 has a window-frame design with two coils 900 wrapped around a return yoke 910. The coils 900 are in a racetrack configuration with 10 turns on each coil 900 and a maximum current of about 100 A, though the number of turns and/or the maximum current can be different in other embodiments. For example, the maximum current can be up to about 150 A to about 200 A in some embodiments. A higher current allows few coil turns and thus a lower inductance, which increases the response speed (e.g., slew rate) of the fast single-axis deflector 90.

The coils 900 can be wound from a hollow conductor material (the bore is the water-cooling channel). A single layer of conductor material can be disposed on each coil which can minimize AC losses. The coils 900 are disposed on opposing sides of the return yoke 910 to deflect the beam in a direction orthogonal to the axis along which the coils 900 are disposed. For example, in FIG. 9 the coils 900 are disposed across from each other along the x axis to deflect the beam parallel to the y axis. Of course, the fast single-axis deflector 90 can also be oriented so that the coils 900 are disposed across from each other along the y axis to deflect the beam parallel to the x axis.

The return yoke 910 has a “window frame” configuration and can be formed of magnetic materials that do not exhibit bulk conductivity (e.g., they have high bulk resistivity) such as ferrites. In some embodiments, the magnetic materials in return yoke 910 have a bulk resistivity of at least about 0.1 Ωm, such as about 0.1 Ωm to about 1×10⁶ Ωm. This high bulk resistivity can significantly inhibit eddy current flow in the return yoke 910. In one example, the return yoke 910 is formed of MN60 (Mn—Zn ferrite), available from Ceramic Magnetics, Inc. of Fairfield, N.J. The return yoke 910 can be about 100 mm long (e.g., along an axis passing into the page of FIG. 9) and the clear gap 920 for the beam can have a pole gap 940 of about 30 mm, about 35 mm, about 40 mm, about 45 mm, or any value or range between any two of the foregoing. The beam can pass through the gap 920 in a non-conducting vacuum pipe, in air, or in helium. In some embodiments, the beam passes through the gap 920 in a non-conductive vessel (e.g., ceramic) with a thin internal conductive coating (e.g., nickel). The pipe or vessel can have a round or a rectangular cross section. The pole gap 940 is defined by the thickness of inward projections 930 on the return yoke 910 which reduce the pole gap 940 for the active axis of deflection (e.g., the y axis in FIG. 9), which can allow for greater deflection without the need for additional coil turns or a larger power supply.

In addition, the return yoke 910 can have a sufficiently-small physical size such that it can fit in the limited space generally available between the end of the beamline 210 and the scan magnet(s) (e.g., X-scan deflector electromagnet 221 and Y-scan deflector electromagnet 222 in scan nozzle 220).

The speed at which an electromagnet can be slewed from one field setting to another can be limited by the available power amplifier voltage, which cannot be made arbitrarily large for reasons of cost, safety, and reliability. The slewing speed of the coil current (which to first order determines the field slewing) is given by dI/dt=V−IR/L. Coil resistance R can be made rather small in an electromagnet, so resistive voltage IR is small and the speed is primarily set by available voltage V and the inductance L. In a specific example, the fast single-axis deflector 90 can have an inductance of about 75 μH to about 250 μH, including about 100 μH, about 125 μH, about 150 μH, about 175 μH, about 200 μH, about 225 μH, or any inductance or inductance range between any two of the foregoing inductances. The fast single-axis deflector 90 can have a resistance R of about 9 mohm and can be powered by a 140 A DC, 350V power amplifier which can be manufactured by International Electric Co. of Helsinki, Finland. In this example, the resistive voltage IR is at most 1.35V and can be almost ignored since the power amplifier can have, for example, 350 V voltage compliance.

In some embodiments, the current control loop of the power amplifier for the fast single-axis deflector 90 can operate at maximum speed (e.g., about 100 kHz) and can have a maximum slew rate (thus minimum ringing or undershoot when settling to a new current setting) when the load is purely resistive, whereas inductance can introduce phase shifts that must be compensated and inevitably can reduce the overall speed of response. As such, reducing the inductance of the fast single-axis deflector 90 (e.g., the electromagnets) increases the overall speed of response.

The relatively small maximum magnetic field strength of the fast single-axis deflector 90 and its relatively small maximum deflection angle enable its operation with minimal hysteresis or eddy currents. The relatively small maximum magnetic field strength also allows the field return yoke 910 to be constructed from a non-conductive magnetic material such as a ferrite material which has negligible eddy currents. Typically, ferrite materials cannot be used in the main scan magnet(s) (e.g., X-scan deflector electromagnet 221 and Y-scan deflector electromagnet 222 in scan nozzle 220) because the yoke magnetic fields are too high for such ferrite materials, which fully saturates them and is undesirable. In some embodiments, the fast single-axis deflector 90 has a maximum magnetic field strength of about 400 Gauss to about 600 Gauss. This is significantly lower than the maximum magnetic field strength of a typical scan magnet, which is about 5,000 Gauss to about 10,000 Gauss. Thus, the maximum magnetic field strength of the fast single-axis deflector 90 can be about 10× to about 25× lower than the maximum magnetic field strength of the main scan magnet(s).

The relatively low magnetic field generated by the fast single-axis deflector 90 can deflect the beam up to an angle of about 2.75 mrad to about 3.75 mrad, including about 3.0 mrad, about 3.25 mrad, about 3.5 mrad, or any angle or range of angles between any two of the foregoing angles, with respect to the trajectory of the beam before it enters the fast single-axis deflector 90. This change in trajectory of the beam can cause the beam to deflect about 6 mm to about 10 mm with respect to the deflection axis at isocenter plane when the fast single-axis deflector 90 is located about 2.3 meters from the isocenter plane. This deflection is sufficient to deflect the beam between most adjacent spots during treatment, as discussed above.

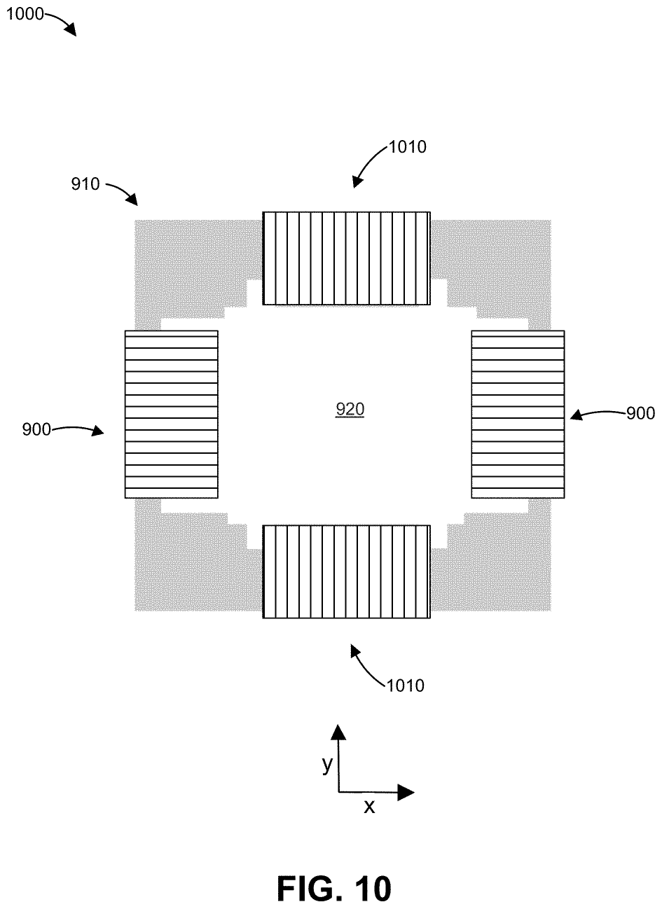

FIG. 10 illustrates an example structure of a fast dual-axis deflector 1000, according to one or more embodiments. The fast dual-axis deflector 1000 is the same as the fast single-axis deflector 90 except that the fast dual-axis deflector 1000 includes a second pair of coils 1010. Coils 1010 are disposed across from each other along the y axis to deflect the beam parallel to the x axis. Thus, fast dual-axis deflector 1000 includes a first pair of coils to deflect the beam along the y axis and a second pair of coils 1010 to deflect the beam parallel to the x axis.

The present disclosure should not be considered limited to the particular embodiments described above, but rather should be understood to cover all aspects of the disclosure as fairly set out in the present disclosure. Various modifications, equivalent processes, as well as numerous structures to which the present disclosure may be applicable, will be readily apparent to those skilled in the art to which the present disclosure is directed upon review of the present disclosure.

What is claimed is:

1. A system for increasing dose delivery efficiency during charged particle beam therapy, comprising:
a charged particle beam generator to generate a charged particle beam;
a transport beamline apparatus comprising beamline deflector magnets that generate magnetic fields to deflect the charged generated particle beam towards a scan nozzle parallel to a reference axis;
the scan nozzle comprising:
a scan electromagnet that generates a scan magnetic field to deflect the charged particle beam along a trajectory to a first target position on an isocenter plane; and
a detector apparatus disposed between the at least one scan magnet and the isocenter plane, the detector apparatus configured to output a signal representing a measured position of the charged particle beam with respect to orthogonal first and second axes, the reference axis orthogonal to the first and second axes; and
a fast deflector electromagnet assembly disposed between the transport beamline apparatus and the scan nozzle, the fast deflector electromagnet assembly configured to (a) receive a first control signal and (b) generate a first magnetic field based on the first control signal, the first magnetic field and the scan magnetic field providing a combined deflection of the charged particle beam to deflect the generated particle beam from a first trajectory corresponding to the first target spot on the isocenter plane to a second trajectory corresponding to a second target spot on the isocenter plane;
wherein the control system comprises a processor, the control system configured to:
receive as an input the first and second spot positions,
determine a trajectory correction by comparing the first and second spot positions, and
generate the first control signal based on the trajectory correction, and
wherein an inductance of the fast deflector electromagnet assembly is lower than an inductance of the scan electromagnet.

2. The system of claim 1, wherein a slew rate of the fast deflector electromagnet assembly is higher than a slew rate of the scan electromagnet.

3. The system of claim 1, wherein the fast deflector electromagnet assembly includes a fast single-axis deflector electromagnet assembly that only deflects the charged particle beam with respect to the first axis.

4. The system of claim 3, wherein the controller is further configured to determine whether the second target spot is located in a preferred direction from the first target spot, the preferred direction parallel to the first axis.

5. The system of claim 1, wherein the control system is further configured to:
determine when a predetermined dose is delivered to the first target spot, and
send the first control signal to the fast electromagnet when the predetermined dose is delivered to the first target spot.

6. The system of claim 5, wherein the control system is further configured to:
generate a second control signal based on the trajectory correction;
determine when the measured position of the charged particle beam corresponds to the second target spot; and
when the measured position of the charged particle beam corresponds to the second target spot:
send the second control signal to the scan electromagnet to adjust the scan magnetic field to deflect a hypothetical charged particle beam from the first target spot to the second target spot, the hypothetical charged particle beam having the first trajectory,
generate a third control signal that adjusts a power to the fast electromagnet such that the charged generated particle beam stays on the second target spot while the scan electromagnet adjusts the scan magnetic field according to the second control signal, and
send the third control signal to the fast electromagnet.

7. The system of claim 6, wherein the third control signal causes the fast deflector electromagnet to transition to an off state when the scan electromagnet has adjusted the scan magnetic field to deflect the hypothetical charged particle beam and the charged particle beam from the first target spot position to the second target spot.

8. The system of claim 1, wherein the fast deflector electromagnet assembly includes a fast dual-axis deflector electromagnet assembly that can deflect the charged particle beam with respect to the first axis, the second axis, or both the first and second axes.

9. The system of claim 1, wherein the fast deflector electromagnet assembly includes a return yoke comprised of a magnetic material having a bulk resistivity of at least about 0.1 Ωm.

10. The system of claim 9, wherein the non-conductive magnetic material comprises a ferrite material.

11. The system of claim 9, wherein the inductance of the fast deflector electromagnet assembly is about 75 μH to about 250 μH.

12. A method for increasing dose delivery efficiency during charged particle beam therapy, the method comprising:
(a) generating a scan magnetic field with a scan electromagnet to deflect a charged particle beam to a first target spot on an isocenter plane;
(b) generating a second magnetic field with a fast deflector electromagnet, the scan magnetic field and the second magnetic field providing a combined deflection of the charged particle beam from a first trajectory corresponding to the first target spot to a second trajectory corresponding to a second target spot on the isocenter plane, the fast deflector electromagnet having a lower inductance than the scan electromagnet, the scan electromagnet disposed between the fast deflector electromagnet and the isocenter plane;
(c) after step (b), simultaneously adjusting the scan magnetic field and the second magnetic field to reduce a contribution of the second magnetic field to the combined deflection of the charged particle beam; and
(d) maintaining the second trajectory of the charged particle beam during step (c).

13. The method of claim 12, wherein step (c) further comprises decreasing a magnitude of the second magnetic field while increasing a magnitude of the scan magnetic field.

14. The method of claim 12, wherein step (c) further comprises increasing a magnitude of the second magnetic field while decreasing a magnitude of the scan magnetic field.

15. The method of claim 12, wherein step (d) further comprises:
detecting a detected position of the charged particle beam with at least one ion chamber detector disposed between the scan electromagnet and the isocenter plane;
determining whether the detected position corresponds to the second target spot; and
when the detected position does not correspond to the second target spot, adjusting the scan magnetic field, the second magnetic field, or both the scan magnetic field and the second magnetic field until the detected position corresponds to the second target spot.

16. The method of claim 12, further comprising:
(e) receiving an irradiation map that includes a location of the first and second target spots; and
(f) determining whether the second target spot is located in a preferred direction from the first target spot, the preferred direction parallel to an axis of deflection of the fast deflector electromagnet.

17. The method of claim 16, wherein the irradiation map includes the location of a third target spot on the isocenter plane, and the method further comprises:
(g) determining whether the third target spot is located in the preferred direction from the second target spot;
(h) when the third target spot is located in the preferred direction from the second target spot, generating a third magnetic field with the fast deflector electromagnet, the scan magnetic field and the third magnetic field providing a second combined deflection of the charged particle beam from the second trajectory corresponding to the second target spot to a third trajectory corresponding to the third target spot; and
(i) when the third target spot is not located in the preferred direction from the second target spot, generating a second scan magnetic field with the scan electromagnet to deflect the charged particle beam from the second target spot to the third target spot.

18. The method of claim 17, further comprising:
(j) after step (h), simultaneously adjusting the scan magnetic field and the third magnetic field to reduce a contribution of the third magnetic field to the second combined deflection; and
(k) maintaining the third trajectory of the charged particle beam during step (j).

19. A system for increasing dose delivery efficiency during charged particle beam therapy, the system comprising:
a scan nozzle comprising:
a scan electromagnet that generates a scan magnetic field to direct the charged particle beam along a trajectory to a target position on an isocenter plane;
a detector apparatus disposed between the scan magnet and the isocenter plane, the detector apparatus outputting a signal representing a measured position of the charged particle beam with respect to orthogonal first and second axes, wherein the first and second axes are orthogonal to a reference axis; and
a fast deflector electromagnet assembly configured to (a) receive a first control signal and (b) generate a first magnetic field based on the first control signal, the first magnetic field and the scan magnetic field providing a combined deflection of the charged particle beam that deflects the charged particle beam from a first trajectory corresponding to a first target spot on the isocenter plane to a second trajectory corresponding to a second target spot on the isocenter plane,
wherein:
the scan nozzle is disposed between the fast deflector electromagnet assembly and the isocenter plane, and
an inductance of the fast deflector electromagnet assembly is lower than an inductance of the scan electromagnet.

20. The system of claim 19, wherein the scan nozzle receives a second control signal and the fast deflector electromagnet assembly receives a third control signal, the second and third control signals causing the scan nozzle and the fast deflector electromagnet assembly to simultaneously adjust the scan magnetic field and the first magnetic field, respectively, to reduce a contribution of the first magnetic to the combined deflection of the charged particle beam.

21. The system of claim 19, wherein the inductance of the fast deflector electromagnet assembly is about 75 μH to about 250 μH and the fast deflector electromagnet assembly includes a return yoke comprised of a ferrite material.
